(19) United States
(12) Patent Application Publication
(10) Pub. No.: US 2026/0201277 A1
(43) Pub. Date: Jul. 16, 2026

(54) PROCESS FOR REGENERATION OF AN EPOXIDATION REACTOR

(71) Applicants: BASF SE, Ludwigshafen am Rhein (DE); Dow Global Technologies LLC, Midland, MI (US)

(72) Inventors: Edward O. MADENJIAN, Midland, MI (US); Kenric A. MARSHALL, Midland, MI (US); Setthawut PIYATANTI, Rayong (TH)

(21) Appl. No.: 19/134,357

(22) PCT Filed: Dec. 1, 2023
(86) PCT No.: PCT/EP2023/083929

§ 371 Date: May 30, 2025

(30) Foreign Application Priority Data
Dec. 2, 2022 (EP) 22211040.5

(51) Int. Cl. C11D 3/36; B01J 38/62; C07D 301/12

(52) U.S. Cl. CPC C11D 3/361; B01J 38/62; C07D 301/12

(57) ABSTRACT

The present invention relates in a first aspect to a process for the regeneration of an epoxidation reactor, the epoxidation reactor having been used in a method for the preparation of an olefin oxide comprising:
(i) providing an organic solvent, an olefin, an epoxidation agent and water to the reactor, which comprises an heterogeneous epoxidation catalyst in an epoxidation zone, so that a reaction mixture comprising olefin, hydrogen peroxide, water and organic solvent is formed;
(ii) subjecting the mixture of (i) in the reactor's epoxidation zone to epoxidation conditions in the presence of the catalyst, thereby obtaining a mixture comprising water, the organic solvent and olefin oxide;
(iii) removing the mixture comprising water, the organic solvent and olefin oxide obtained in (ii) from the reactor;
whereby a precipitate is deposited in the reactor;
said process for the regeneration comprising:
(a) stopping providing organic solvent, olefin, epoxidation agent and water to the reactor;
(b) introducing a liquid aqueous system into the reactor, wherein the liquid aqueous system comprises a chelating agent, which comprises diphosphonic acid of formula (I)

(OH)₂(O═)P—CR¹R²—P(═O)(OH)₂ (I),
wherein R¹ and R² are independently selected from the group consisting of hydrogen atom, hydroxyl group and C1 to C5 alkyl group, wherein R¹ is preferably a hydroxyl group and R² is preferably a methyl group (1-hydroxy ethylidene-1,1-diphosphonic acid, HEDP).

A second aspect of the invention is related to a combined process for preparation of an olefin oxide comprising a preparation stage and a stage of regeneration of the epoxidation reactor, the preparation stage comprising steps (i), (ii) and (iii), whereby a precipitate is deposited in the reactor; the regeneration stage comprising steps (a) and (b).

The present invention relates in a first aspect to a process for the regeneration of an epoxidation reactor, the epoxidation reactor having been used in a method for the preparation of an olefin oxide comprising: (i) providing an organic solvent, an olefin, an epoxidation agent and water to the reactor, which comprises an heterogeneous epoxidation catalyst in an epoxidation zone, so that a reaction mixture comprising olefin, hydrogen peroxide, water and organic solvent is formed; (ii) subjecting the mixture of (i) in the reactor's epoxidation zone to epoxidation conditions in the presence of the catalyst, thereby obtaining a mixture comprising water, the organic solvent and olefin oxide; (iii) removing the mixture comprising water, the organic solvent and olefin oxide obtained in (ii) from the reactor; whereby a precipitate is deposited in the reactor;
said process for the regeneration comprising: (a) stopping providing organic solvent, olefin, epoxidation agent and water to the reactor; and (b) introducing a liquid aqueous system into the reactor, wherein the liquid aqueous system comprises a chelating agent, which comprises at least three oxygen atom(s) and optionally one or more nitrogen atom(s) in its structure and which is capable of forming or forms at least three coordinative bonds via said at least three oxygen atom(s) and optionally via its one or more nitrogen atom(s) to a metal cation when forming a chelate complex with said metal cation. A second aspect of the invention is related to a combined process for preparation of an olefin oxide comprising a preparation stage and a stage of regeneration of the epoxidation reactor, the preparation stage comprising steps (i), (ii) and (iii), whereby a precipitate is deposited in the reactor; the regeneration stage comprising steps (a) and (b).

Propylene oxide (PO) is one of the most important chemical intermediates in industry. It represents the starting compound for a broad spectrum of products, such as foams, solvents or de-icing agents. Traditionally, PO is produced via the chlorohydrin process, which is still in use today, as well as the oxirane method. The development of catalysts based on zeolitic materials having a framework structure comprising Si, O, and Ti, such as titanium silicalite-1, together with the improved availability of large quantities of hydrogen peroxide enabled the large-scale implementation of the co-product-free HPPO technology. This new process enables PO to be produced with excellent yields and selectivities.

The HPPO process produces propylene oxide from propylene and hydrogen peroxide in aqueous organic solvents with zeolitic materials having a framework structure comprising Si, O, and Ti as catalysts. In one constellation, methanol is used as the solvent, typically in combination with a zeolitic material having a framework structure comprising Si, O, and Ti of framework type MFI (titanium silicalite-1, TS-1) as catalyst. In another constellation, acetonitrile is used as the solvent, typically in combination with a zeolitic material having a framework structure comprising Si, O, and Ti of framework type MWW (TiMWW) as catalyst.

In the production of propylene oxide according to the HPPO process, precipitates are known to deposit over time in certain critical areas of the process equipment. In particular, deposition of these precipitates at the entrance of the epoxidation reactors obstructs the flow of feeds to these reactors. This leads to increased pressure drop across the system and maldistribution of flow. Higher pressure drop across the reactors can lead to decreased throughput (production) from the process, and maldistribution of flow can lead to loss of selectivity to the desired product, as some tubes will experience high residence time/low conversion while others will experience low residence time/high conversion when compared to the process conditions which yield optimal performance.

It is assumed that the precipitates comprise deposited salts, which are derived from a combination of components intentionally added to the process to enhance reactor performance, stabilizers present in the hydrogen peroxide feed, corrosion products, as well as other sources of metals. The salts, once deposited, are difficult to remove from the process equipment. Jetting or blasting of the deposits requires clearing and entry into the areas to be cleaned, which leads to extended down time of the reactors as well as risks damage to the catalyst. This technique also cannot remove precipitates/salts deposited on, in, or around the catalyst particles themselves. Chemical methods such as solvation/dissolution can be used in principle without requiring clearing and entry or causing physical damage to the catalyst. However, these precipitates/salts are relatively insoluble. WO 2016/128538 A1, for example, describes a process for the regeneration of a catalyst comprising a titanium containing zeolite as catalytically active material, wherein the catalyst is washed with a liquid aqueous system, which consists mostly of water. However, water is normally ineffective as a solvent for removing the precipitates/deposited salts. Harsh cleaning agents can damage the catalyst or corrode process equipment and are not desirable. WO 2015/010994 A1, for example, discloses a regeneration process, wherein the catalyst is washed with a liquid aqueous system. The liquid aqueous system employed consists mostly of water and addition of acids is to be avoided, i.e. the liquid aqueous system has to have a basic pH value. However, also this process regime turned out to be not able to dissolve highly insoluble, complex salts in a satisfying manner.

The object underlying the present invention was thus the provision of an improved process for dissolving highly insoluble, complex salts without damaging the catalyst or process equipment.

### 1^{st} Aspect—Regeneration Process

In a first aspect, the object was solved by a process for the regeneration of an epoxidation reactor, the epoxidation reactor having been used in a method for the preparation of an olefin oxide comprising:
(i) providing an organic solvent, an olefin, an epoxidation agent and water to the reactor, which comprises an heterogeneous epoxidation catalyst in an epoxidation zone, so that a reaction mixture comprising olefin, hydrogen peroxide, water and organic solvent is formed;
(ii) subjecting the mixture of (i) in the reactor's epoxidation zone to epoxidation conditions in the presence of the catalyst, thereby obtaining a mixture comprising water, the organic solvent and olefin oxide;
(iii) removing the mixture comprising water, the organic solvent and olefin oxide obtained in (ii) from the reactor;
whereby a precipitate is deposited in the reactor;
said process for the regeneration comprising:
(a) stopping providing organic solvent, olefin, epoxidation agent and water to the reactor;
(b) introducing a liquid aqueous system into the reactor, wherein the liquid aqueous system comprises a chelating agent, which comprises at least three oxygen atom(s) and optionally one or more nitrogen atom(s) in its structure and which is capable of forming or forms at least three coordinative bonds via said at least three oxygen atom(s) and optionally via its one or more nitrogen atom(s) to a metal cation when forming a chelate complex with said metal cation.

In some embodiments, the chelating agent has a log K value with respect to trivalent iron cation (Fe³⁺) of at least 10, preferably in the range of from 11 to 30, more preferably in the range of from 15 to 30, wherein these values are based on measurement in a constant temperature cell at a temperature in the range of from 20 to 25° C., at a constant ionic strength I of 0.1 mol l⁻¹, established by a suitable salt of an alkaline metal cation such as KNO₃.

In some embodiments, the chelating agent, which comprises at least three oxygen atom(s) and optionally one or more nitrogen atom(s) in its structure is capable of forming or forms, preferably forms, in the range of from 3 to 8 coordinative bonds via said one or more nitrogen atom(s) and/or one or more oxygen atom(s) to a metal cation when forming a chelate complex with said metal cation. The expression “capable of forming or forms, preferably forms, in the range of from 3 to 8 coordinative bonds via said one or more nitrogen atom(s) and/or one or more oxygen atom(s) to a metal cation” means that the chelating agents forms or is capable of forming at least 3 coordinative bonds to one (1) metal cation. In case that the chelating agent is capable of forming more than 3 coordinative bonds, these more than three coordinative bonds might be directed to the same metal cation as the initial three coordinative bonds but may also be direct to one or more further metal cation(s). For example, if the chelating agent is EDTA (or a complete or partial salt thereof), the chelating agent is capable of forming overall six coordinative bonds—three of these six coordinative bonds must be directed or directable to one single metal cation. Contrary to the preferred chelating agents listed in the following, pyrophosphate, for instance in the form of sodium acid pyrophosphate (SAPP), while having more coordinative sites, is, due to its structure, only capable of forming 2 coordinative bonds to one metal cation.

In some embodiments, the chelating agent is selected from the group consisting of
ethylene diamine tetraacetic acid (EDTA),
diethylene triamine pentaacetic acid (DTPA),
N-(hydroxyethyl) ethylene diamine triacetic acid (HEDTA),
N-(1-carboxyethyl)-imino diacetic acid (MGDA),
nitrilotriacetic acid (NTA),
L-glutamic acid-N,N-diacetic acid (GLDA),
aminotris(methylenephosphonic acid) (AMTP),
ethanol diglycinic acid (EDG),
diphosphonic acid of formula (I)

(OH)₂(O═)P—CR¹R²—P(═O)(OH)₂ (I),
wherein R¹ and R² are independently selected from the group consisting of hydrogen atom, hydroxyl group and C1 to C5 alkyl group,
   and mixtures of two or more of these chelating agents.

In some embodiments, the chelating agent is selected from the group consisting of EDTA, DTPA, HEDTA, MGDA, NTA, AMTP, diphosphonic acid of formula (I) as indicated above and mixtures of two or more of these chelating agents.

In some preferred embodiments, the chelating agent comprises, preferably is, a diphosphonic acid of formula (I), wherein R¹ is preferably a hydroxyl group and R² is preferably a methyl group (1-hydroxy ethylidene-1,1-diphosphonic acid, HEDP).

The chelating agent is used in its protonated form, or in an at least partially deprotonated form, for example, in the form of a partial or complete salt, i.e. one of its corresponding anions with a suitable cation to compensate the anion's charge. In some preferred embodiments, the chelating agent is used in its fully protonated form.

Compared to neat water and also to washing solutions containing sodium acid pyrophosphate (SAPP), use of a liquid aqueous system comprising a chelating agent as defined above, especially a diphosphonic acid of formula (I), significantly improved dissolution of the precipitates. It could further be shown that the liquid aqueous system comprising a chelating agent as defined above, especially a diphosphonic acid of formula (I), did not damage the catalyst in any way or impair the catalyst's performance. Furthermore, it was shown that the process vessels were not damaged by the liquid aqueous system comprising a chelating agent as defined above, especially a diphosphonic acid of formula (I).

In some preferred embodiments of the process for the regeneration of an epoxidation system the liquid aqueous system used in (b) comprises in the range of from 0.01 to 50 weight-%, preferably in the range of from 0.02 to 25 weight-%, more preferably in the range of from 0.05 to 10 weight-% of the chelating agent, preferably of the diphosphonic acid, more preferably HEDP, more preferably in the range of from 0.1 to 5 weight-% of the chelating agent, preferably of the diphosphonic acid, more preferably HEDP, each based on the total weight of the liquid aqueous system being 100 weight-%.

In some preferred embodiments of the process for the regeneration of an epoxidation system step (b) comprises:
(b.1) introducing a first portion of liquid aqueous system, which comprises a chelating agent, into the reactor;
(b.2) removing the first portion of the liquid aqueous system at least partially from the reactor.

In some preferred embodiments of the process for the regeneration of an epoxidation system step (b) comprises:
(b.1) introducing a first portion of liquid aqueous system, which comprises a chelating agent, into the reactor;
(b.2) removing the first portion of the liquid aqueous system at least partially from the reactor;
(b.3) optionally introducing a further portion of liquid aqueous system, which comprises chelating agent, into the reactor;
(b.4) optionally removing the further portion of the liquid aqueous system at least partially from the reactor;
wherein steps (b.3) and (b.4) are optionally repeated for 1-10 times.

In some preferred embodiments of the process for the regeneration of an epoxidation system introducing the liquid aqueous system in step(s) (b) and/or (b.1) and/or (b.3) is carried out with a gaseous stream passing through the liquid aqueous system, wherein the gaseous stream preferably comprises at least 90 volume-%, more preferably at least 95 volume-%, more preferably at least 98 volume-% of an inert gas, based on the total volume of the gaseous stream. An “Inert gas” is preferably selected from the group consisting of helium, neon, argon, krypton, xenon, nitrogen and mixtures of two or more of these inert gases, more preferably the inert gas comprises at least 90 volume-%, more preferably at least 95 volume-%, more preferably at least 98 volume-% of nitrogen.

In some preferred embodiments of the process for the regeneration of an epoxidation system introducing the liquid aqueous system in step(s) (b) and/or (b.1) and/or (b.3) is carried out at a temperature in the reactor in the range of from 0.1° C. to the boiling temperature of the liquid aqueous system, preferably in the range of from 0.5 to 90° C., more preferably in the range of from 5 to 50° C., more preferably in the range of from 10 to 40° C.

In some preferred embodiments of the process for the regeneration of an epoxidation system introducing the liquid aqueous system in step(s) (b) and/or (b.1) and/or (b.3) is carried out at a pressure in the reactor in the range of from 0.05 to 5.0 MPa, preferably in the range of from 0.08 to 1.0 MPa, more preferably in the range of from 0.1 to 0.15 MPa.

In some preferred embodiments of the process for the regeneration of an epoxidation system the reactor comprises one or more vertically arranged tube(s) each of a specific length (height) having a bottom end and a top end, wherein the catalyst is present in the tube(s) between a position H1 and a position H2, wherein H2 is higher up in the tube(s) than H1, and introducing the liquid aqueous system in step (b) and/or (b.1) and/or (b.3) is carried out so that the liquid level within the tube(s) is at a high of at least H2, preferably at a height in the range between H1 and H2, more preferably at a height in the range of from 80 to 5% below H2, more preferably at a height in the range of from 70 to 10% below H2, more preferably at a height in the range of from 60 to 20% below H2, more preferably at a height in the range of from 50 to 25% below H2, more preferably at a height in the range of from 40 to 30% below H2, each in relation to the distance between H1 and H2 being 100%. In other words, introducing the liquid aqueous system in step (b) and/or (b.1) and/or (b.3) is carried out in these preferred embodiments so that at least all the catalyst is immersed in the liquid aqueous system, preferably all the catalyst is immersed in the liquid aqueous system, more preferably in the range of from 20 to 95% of the catalyst are immersed in the liquid aqueous system, more preferably in the range of from 30 to 90% of the catalyst are immersed in the liquid aqueous system, more preferably in the range of from 40 to 80% of the catalyst are immersed in the liquid aqueous system, more preferably in the range of from 50 to 75% of the catalyst are immersed in the liquid aqueous system.

The term “vertically” comprises any arrangement that is about vertically, i.e. vertically also comprises arrangements in which the tube is placed at an angle of ±45° with respect to the vertical axis. Preferably, the tubes are arranged in a tube sheet, which is present within the lower part of reactor. The catalyst, i.e. the catalyst filing, is preferably kept in place within the tubes by a catalyst support, which is present in the tube in the region between bottom end of each tube and H1. The catalyst support is permeable for gaseous and liquid components, such as the liquid aqueous system but maintains the catalyst in place within the tube. In some embodiments, the position of the tube sheet is higher than the position of the catalyst support, each relative to the height of each tube. In some embodiments, there is also an inert material positioned in the tubes between catalyst support and H1. The inert material is permeable for gaseous and liquid components, such as the liquid aqueous system.

In some preferred embodiments of the process for the regeneration of an epoxidation system after step (b.2), the amount of precipitate dissolved in the liquid aqueous system, which is removed at least partially from the reactor, is determined. More preferably, the amount of precipitate dissolved in the liquid aqueous system determined after step (b.2) is further set into relation to the amount of catalyst. If the percentage value of dissolved precipitate per catalyst is ≤1%, more preferably ≤0.9%, more preferably ≤0.8%, after step (b.2), no further steps (b.3), (b.4) are conducted in some preferred embodiments. If the percentage value of dissolved precipitate per catalyst is above 1%, steps (b.3) and (b.4) are carried out and optionally repeated for 1 to 10 times are conducted in some preferred embodiments, wherein preferably after each step (b.4) the amount of precipitate dissolved in the liquid aqueous system, which is removed at least partially from the reactor, is determined and steps (b.3) and (b.4) are repeated until the percentage value of dissolved precipitate per catalyst is ≤1%, more preferably ≤0.9%, more preferably ≤0.8%.

In some embodiments, for determining the amount of precipitate dissolved in the liquid aqueous system, initially the concentration of the various components in the liquid aqueous system prior to its use is determined as a baseline (initial concentration, c₀), then the amount of precipitate dissolved in the liquid aqueous system after removal from the reactor is determined (concentration cₓ) and by forming the difference between cₓ and c₀, the amount of precipitate dissolved in the liquid aqueous system is determined. Preferably, for determining the concentration of the various components in the liquid aqueous system, metals are investigated, preferably one or more metal(s) from the group consisting of aluminum (Al), silicon (Si), sodium (Na), magnesium (Mg), potassium (K), calcium (Ca), titanium (Ti), chromium (Cr), manganese (Mn), iron (Fe), nickel (Ni), copper (Cu) and zinc (Zn), and also the concentration of phosphor (P).

The indication of “metal” or the naming of specific metals such as aluminum or iron means that these metals are not present in elementary form, i.e. with their oxidation state being zero, but rather in the form of metal cations, wherein the positive charge is compensated by one or more anions. The same applies for other chemical elements such as P, which is normally present as an oxyanion, for example, phosphate (PO₄³⁻) or hydrogen phosphate (HPO₄²⁻) or pyrophosphate (H₂P₂O₇²⁻).

Means for determining the concentration of metal (cations) as well as concentrations of other chemical elements and their cations or anions are well known to the skilled person, such as evaporating a sample to dryness and then determining the amount of remaining solid by weighting. In some preferred embodiments, Inductively Coupled Plasma-Optical Emission Spectrometry (ICP-OES) is used for the analysis, i.e. the determination of the concentration, preferably directly in a liquid sample. If ICP-OES is used, for each metal a suitable anion is chosen such as, preferably, a suitable oxygen- and/or phosphor-based anion, and, based on the amount of metal, oxygen and/or phosphor, the weight of the precipitated salt is calculated. Exemplary salts used for the analysis and calculation are Al₂(H₂P₂O₇)₃, SiO₂, Mg₃(PO₄)₂, K₂HPO₄, Ca₃(PO₄)₂, TiO₂, Cr₂O₃, Fe₂(H₂P₂O₇)₃, NiO; whereas sodium (Na) is in some embodiments calculated directly based on the Na concentration.

### 2^{nd} Aspect—Combined Process

The invention relates in a second aspect to a combined process for preparation of an olefin oxide comprising a preparation stage and a stage of regeneration of the epoxidation reactor, the preparation stage comprising:
(i) providing an organic solvent, an olefin, an epoxidation agent and water to the reactor comprising a heterogeneous epoxidation catalyst in an epoxidation zone, so that a reaction mixture comprising olefin, hydrogen peroxide, water and organic solvent is formed;
(ii) subjecting the mixture formed in (i) in the epoxidation reactor to epoxidation conditions in the presence of the catalyst, thereby obtaining a mixture comprising water, the organic solvent and olefin oxide;
(iii) removing the mixture comprising water, the organic solvent and olefin oxide obtained in (ii) from the reactor;
whereby a precipitate is deposited in the reactor;
the regeneration stage comprising:
(a) stopping introducing the mixture of (i) into the reactor;
(b) introducing a liquid aqueous system into the reactor, wherein the liquid aqueous system comprises a chelating agent, which comprises at least three oxygen atom(s) and optionally one or more nitrogen atom(s) in its structure and which is capable of forming or forms at least three coordinative bonds via said at least three oxygen atom(s) and optionally via its one or more nitrogen atom(s) to a metal cation when forming a chelate complex with said metal cation.

In some preferred embodiments of the combined process for preparation of an olefin oxide, which comprises a preparation stage and a stage of regeneration of the epoxidation reactor, the chelating agent has a log K value with respect to trivalent iron cation (Fe³⁺) of at least 10, preferably in the range of from 11 to 30, more preferably in the range of from 15 to 30, wherein these values are based on measurement in a constant temperature cell at a temperature in the range of from 20 to 25° C., at a constant ionic strength I of 0.1 mol l⁻¹, established by a suitable salt of an alkaline metal cation such as KNO₃.

In some preferred embodiments of the combined process for preparation of an olefin oxide, which comprises a preparation stage and a stage of regeneration of the epoxidation reactor, the chelating agent, which comprises at least three oxygen atom(s) and optionally one or more nitrogen atom(s) in its structure is capable of forming or forms, preferably forms, in the range of from 3 to 8 coordinative bonds via said one or more nitrogen atom(s) and/or one or more oxygen atom(s) to a metal cation when forming a chelate complex with said metal cation. The expression “capable of forming or forms, preferably forms, in the range of from 3 to 8 coordinative bonds via said one or more nitrogen atom(s) and/or one or more oxygen atom(s) to a metal cation” means that the chelating agents forms or is capable of forming at least 3 coordinative bonds to one (1) metal cation. In case that the chelating agent is capable of forming more than 3 coordinative bonds, these more than three coordinative bonds might be directed to the same metal cation as the initial three coordinative bonds but may also be direct to one or more further metal cation(s). For example, if the chelating agent is EDTA (or a (partial) salt thereof), the chelating agent is capable of forming overall six coordinative bonds—three of these six coordinative bonds must be directed or directable to one single metal cation.

In some preferred embodiments of the combined process for preparation of an olefin oxide, which comprises a preparation stage and a stage of regeneration of the epoxidation reactor, the chelating agent is selected from the group consisting of
ethylene diamine tetraacetic acid (EDTA),
diethylene triamine pentaacetic acid (DTPA),
N-(hydroxyethyl) ethylene diamine triacetic acid (HEDTA),
N-(1-carboxyethyl)-imino diacetic acid (MGDA),
nitrilotriacetic acid (NTA),
L-glutamic acid-N,N-diacetic acid (GLDA),
aminotris(methylenephosphonic acid) (AMTP),
ethanol diglycinic acid (EDG),
diphosphonic acid of formula (I)

(OH)₂(O═)P—CR¹R²—P(═O)(OH)₂ (I),
wherein R¹ and R² are independently selected from the group consisting of hydrogen atom, hydroxyl group and C1 to C5 alkyl group,
   and mixtures of two or more of these chelating agents.

In some preferred embodiments of the combined process for preparation of an olefin oxide, which comprises a preparation stage and a stage of regeneration of the epoxidation reactor, the chelating agent is selected from the group consisting of EDTA, DTPA, HEDTA, MGDA, NTA, AMTP, diphosphonic acid of formula (I) as indicated above and mixtures of two or more of these chelating agents.

In some preferred embodiments of the combined process for preparation of an olefin oxide, which comprises a preparation stage and a stage of regeneration of the epoxidation reactor, the chelating agent is a diphosphonic acid of formula (I),
wherein R¹ is preferably a hydroxyl group and R² is preferably a methyl group (1-hydroxy ethylidene-1,1-diphosphonic acid, HEDP).

The chelating agent is used in its protonated form, or in an at least partially deprotonated form, for example, in the form of a salt, i.e. one of its corresponding anions with a suitable cation to compensate the anion's charge. In some preferred embodiments, the chelating agent is used in its fully protonated form.

In some preferred embodiments of the combined process for preparation of an olefin oxide, which comprises a preparation stage and a stage of regeneration of the epoxidation reactor, the liquid aqueous system used in (b) comprises in the range of from 0.01 to 50 weight-%, preferably in the range of from 0.02 to 25 weight-%, more preferably in the range of from 0.05 to 10 weight-% of chelating agent, preferably diphosphonic acid of formula (I), more preferably HEDP, more preferably in the range of from 0.1 to 5 weight-% of chelating agent, preferably diphosphonic acid, more preferably HEDP, each based on the total weight of the liquid aqueous system being 100 weight-%.

In some preferred embodiments of the combined process for preparation of an olefin oxide, which comprises a preparation stage and a stage of regeneration of the epoxidation reactor:
(b.1) introducing a first portion of liquid aqueous system, which comprises a chelating agent, into the reactor;
(b.2) removing the first portion of the liquid aqueous system at least partially from the reactor.

In some preferred embodiments of the combined process for preparation of an olefin oxide, which comprises a preparation stage and a stage of regeneration of the epoxidation reactor, step (b) comprises:
(b.1) introducing a first portion of liquid aqueous system, which comprises a chelating agent, into the reactor;
(b.2) removing the first portion of the liquid aqueous system at least partially from the reactor;
(b.3) optionally introducing a further portion of liquid aqueous system, which comprises chelating agent, into the reactor;
(b.4) optionally removing the further portion of the liquid aqueous system at least partially from the reactor; wherein steps (b.3) and (b.4) are optionally repeated for 1-10 times.

In some preferred embodiments of the combined process for preparation of an olefin oxide, which comprises a preparation stage and a stage of regeneration of the epoxidation reactor, introducing the liquid aqueous system in step(s) (b) and/or (b.1) and/or (b.3) is carried out with a gaseous stream passing through the liquid aqueous system, wherein the gaseous stream preferably comprises at least 90 volume-%, more preferably at least 95 volume-%, more preferably at least 98 volume-% of an inert gas, based on the total volume of the gaseous stream. An “Inert gas” is preferably selected from the group consisting of helium, neon, argon, krypton, xenon, nitrogen and mixtures of two or more of these inert gases, more preferably the inert gas comprises at least 90 volume-%, more preferably at least 95 volume-%, more preferably at least 98 volume-% of nitrogen.

In some preferred embodiments of the combined process for preparation of an olefin oxide, which comprises a preparation stage and a stage of regeneration of the epoxidation reactor, introducing the liquid aqueous system in step(s) (b) and/or (b.1) and/or (b.3) is carried out at a temperature in the reactor in the range of from 0.1° C. to the boiling temperature of the liquid aqueous system, preferably in the range of from 0.5 to 90° C., more preferably in the range of from 5 to 50° C., more preferably in the range of from 10 to 40° C.

In some preferred embodiments of the combined process for preparation of an olefin oxide, which comprises a preparation stage and a stage of regeneration of the epoxidation reactor, introducing the liquid aqueous system in step(s) (b) and/or (b.1) and/or (b.3) is carried out at a pressure in the reactor in the range of from 0.05 to 5.0 MPa, preferably in the range of from 0.08 to 1.0 MPa, more preferably in the range of from 0.1 to 0.15 MPa.

In some preferred embodiments of the combined process for preparation of an olefin oxide, which comprises a preparation stage and a stage of regeneration of the epoxidation reactor, the reactor comprises one or more vertically arranged tube(s) each of a specific length (height) having a bottom end and a top end, wherein the catalyst is present in the tube(s) between a position H1 and a position H2, wherein H2 is higher up in the tube(s) than H1, and introducing the liquid aqueous system in step (b) and/or (b.1) and/or (b.3) is carried out so that the liquid level within the tube(s) is at a height of at least H2, preferably at a height in the range between H1 and H2, more preferably at a height in the range of from 80 to 5% below H2, more preferably at a height in the range of from 70 to 10% below H2, more preferably at a height in the range of from 60 to 20% below H2, more preferably at a height in the range of from 50 to 25% below H2, more preferably at a height in the range of from 40 to 30% below H2, each in relation to the distance between H1 and H2 being 100%. In other words, introducing the liquid aqueous system in step (b) and/or (b.1) and/or (b.3) is carried out in these preferred embodiments so that at least all the catalyst is immersed in the liquid aqueous system, preferably all the catalyst is immersed in the liquid aqueous system, more preferably in the range of from 20 to 95% of the catalyst are immersed in the liquid aqueous system, more preferably in the range of from 30 to 90% of the catalyst are immersed in the liquid aqueous system, more preferably in the range of from 40 to 80% of the catalyst are immersed in the liquid aqueous system, more preferably in the range of from 50 to 75% of the catalyst are immersed in the liquid aqueous system.

The term “vertically” comprises any arrangement that is about vertically, i.e. vertically also comprises arrangements in which the tube is placed at an angle of ±45° with respect to the vertical axis. Preferably, the tubes are arranged in a tube sheet, which is present within the lower part of reactor. The catalyst, i.e. the catalyst filing, is preferably kept in place within the tubes by a catalyst support, which is present in the tube in the region between bottom end of each tube and H1. The catalyst support is permeable for gaseous and liquid components, such as the liquid aqueous system but maintains the catalyst in place within the tube. In some embodiments, the position of the tube sheet is higher than the position of the catalyst support, each relative to the height of each tube.

In some preferred embodiments of the combined process for preparation of an olefin oxide, which comprises a preparation stage and a stage of regeneration of the epoxidation reactor, after step (b.2), the amount of precipitate dissolved in the liquid aqueous system, which is removed at least partially from the reactor, is determined. More preferably, the amount of precipitate dissolved in the liquid aqueous system determined after step (b.2) is further set into relation to the amount of catalyst. If the percentage value of dissolved precipitate per catalyst is ≤1%, more preferably ≤0.9%, more preferably ≤0.8%, after step (b.2), no further steps (b.3), (b.4) are conducted in some preferred embodiments. If the percentage value of dissolved precipitate per catalyst is above 1%, steps (b.3) and (b.4) are carried out and optionally repeated for 1 to 10 times are conducted in some preferred embodiments, wherein preferably after each step (b.4) the amount of precipitate dissolved in the liquid aqueous system, which is removed at least partially from the reactor, is determined and steps (b.3) and (b.4) are repeated until the percentage value of dissolved precipitate per catalyst is ≤1%, more preferably ≤0.9%, more preferably ≤0.8%. Regarding determination of the amount of precipitate dissolved in the liquid aqueous, the same applies as disclosed above in the first section.

### Epoxidation Agent

In some preferred embodiments of the process for the regeneration of an epoxidation reactor of the first aspect or the combined process for preparation of an olefin oxide according to the second aspect, the epoxidation agent is hydrogen peroxide, wherein the hydrogen peroxide is preferably provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484;
and/or
wherein the hydrogen peroxide has a pH in the range of from 0 to 3.0, preferably in the range of from 0.1 to 2.5, more preferably in the range of from 0.5 to 2.3, determined with a pH sensitive glass electrode according to AM7160;
and/or
wherein the hydrogen peroxide comprises from 20 to 85 weight-%, preferably from 30 to 75 weight-%, more preferably from 40 to 70 weight-% of hydrogen peroxide relative to the total weight of the aqueous hydrogen peroxide solution;
and/or
wherein the hydrogen peroxide is obtained or obtainable from an anthraquinone process.

### Organic Solvent

In some preferred embodiments of the process for the regeneration of an epoxidation reactor of the first aspect or the combined process for preparation of an olefin oxide according to the second aspect, the organic solvent is an organic epoxidation solvent, preferably the organic solvent is selected from the group consisting of alcohol, acetonitrile, propionitrile and mixtures of two or more thereof; more preferred selected from the group consisting of alcohol, acetonitrile and mixtures of alcohol and acetonitrile; more preferred the organic solvent comprises at least an alcohol, wherein the alcohol is preferably a C1 to C5 mono alcohol or a mixture of two or more C1 to C5 alcohols, more preferred the alcohol comprises at least methanol.

### Olefin

In some preferred embodiments of the process for the regeneration of an epoxidation reactor of the first aspect or the combined process for preparation of an olefin oxide according to the second aspect, the olefin is a C2-C10 alkene, preferably a C2-C5 alkene, more preferably a C2-C4 alkene, more preferably ethylene (C2 alkene) or propylene (C3 alkene), more preferably propylene (C3 alkene).

### Additives/Buffers

In some preferred embodiments of the process for the regeneration of an epoxidation reactor of the first aspect or the combined process for preparation of an olefin oxide according to the second aspect, the mixture formed in (i) further comprise an additive, preferably selected from the group consisting of potassium salt, ammonia, ammonium salt, etidronic acid, salt of etidronic acid, and mixtures of two or more thereof; wherein the additive is selected from the group consisting of potassium dihydrogen phosphate, dipotassium hydrogen phosphate, potassium formate, potassium acetate, potassium hydrogen carbonate, etidronic acid, ammonium dihydrogen phosphate, diammonium hydrogen phosphate, ammonia and mixtures of two or more thereof, preferably form the group consisting pf potassium dihydrogen phosphate, dipotassium hydrogen phosphate, etidronic acid, ammonia and mixtures of two or more thereof.

### Catalyst

In some preferred embodiments of the process for the regeneration of an epoxidation reactor of the first aspect or the combined process for preparation of an olefin oxide according to the second aspect, the catalyst comprising a titanium containing zeolite has a framework structure comprising Si, O, and Ti.

In some preferred embodiments of the process for the regeneration of an epoxidation reactor of the first aspect or the combined process for preparation of an olefin oxide according to the second aspect, the catalyst comprising a titanium containing zeolite comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the titanium containing zeolite.

In some preferred embodiments of the process for the regeneration of an epoxidation reactor of the first aspect or the combined process for preparation of an olefin oxide according to the second aspect, the titanium containing zeolite having a framework structure comprising Si, O, and Ti comprised in the titanium containing zeolite has ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the titanium containing zeolite having a framework structure comprising Si, O, and Ti is a titanium containing zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably an MFI framework type, or an MWW framework type; more preferred the titanium containing zeolite having a framework structure comprising Si, O, and Ti has framework type MFI; more preferably the zeolitic material having a framework structure comprising Si, O, and Ti is a titanium silicalite-1 (TS-1).

In some preferred embodiments of the process for the regeneration of an epoxidation reactor of the first aspect or the combined process for preparation of an olefin oxide according to the second aspect, the catalyst comprising a titanium containing zeolite further comprises a binder.

In some preferred embodiments of the process for the regeneration of an epoxidation reactor of the first aspect or the combined process for preparation of an olefin oxide according to the second aspect, the catalyst comprising a titanium containing zeolite is in the form of a molding, preferably in the form of an extrudate or a granule.

In some preferred embodiments of the process for the regeneration of an epoxidation reactor of the first aspect or the combined process for preparation of an olefin oxide according to the second aspect, from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the titanium containing zeolite and the binder.

In some preferred embodiments of the process for the regeneration of an epoxidation reactor of the first aspect or the combined process for preparation of an olefin oxide according to the second aspect, from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.

In some preferred embodiments of the process for the regeneration of an epoxidation reactor of the first aspect or the combined process for preparation of an olefin oxide according to the second aspect, the catalyst comprising a titanium containing zeolite, preferably the molding, comprises the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the catalyst comprising a titanium containing zeolite preferably based on the total weight of the molding and/or wherein the catalyst comprising a titanium containing zeolite, preferably the molding, comprises the titanium containing zeolite in an amount in the range of from 10 to 98 weight-%, preferably in the range of from 30 to 95 weight-%, more preferred in the in the range of from 50 to 90 weight-%, more preferred in the range of from 70 to 85 weight-%, more preferred in the range of from 75 to 80 weight-%, based on the total weight of the catalyst comprising a titanium containing zeolite, preferably based on the total weight of the molding.

In some preferred embodiments of the process for the regeneration of an epoxidation reactor of the first aspect or the combined process for preparation of an olefin oxide according to the second aspect, at least three individual feed streams are provided in (i), at least one of which is the feed with which the organic solvent is provided to the reactor, at least one of which is the feed with which the olefin is provided to the reactor and at least one of which is the feed with which the hydrogen peroxide and the water are provided to the reactor, wherein optionally, at least one further individual stream is provided to the reactor, which is the feed with which an additive is provided to the reactor.

The individual feed streams can be combined and introduced into the reactor as one single feed stream or as combined feed streams such as, for example, a feed stream containing organic solvent hydrogen peroxide and water and a feed stream containing olefin, or a feed stream containing organic solvent and olefin and a feed stream containing hydrogen peroxide and water, or a feed stream containing organic solvent and a feed stream containing olefin and a feed stream containing hydrogen peroxide and water. The optional additive can be added as separate feed stream or can be mixed to one or more of the above-described feed streams. If more than one feed stream is employed, the individual feed streams are either mixed before they are introduced in the reactor or suitably mixed after having been introduced into the reactor.

In some preferred embodiments of the process for the regeneration of an epoxidation reactor of the first aspect or the combined process for preparation of an olefin oxide according to the second aspect, organic solvent, hydrogen peroxide, water olefin and optional additive are mixed before entering the reactor, so that a single feed stream comprising organic solvent, hydrogen peroxide, water olefin and optional additive is provided to the reactor.

Preferably, the respective individual streams are suitably mixed to obtain a reaction feed, which consists of at least one liquid phase. Even more preferably, the individual streams are suitably mixed to obtain a feed stream, which consists of one liquid phase.

In some preferred embodiments of the process for the regeneration of an epoxidation reactor of the first aspect or the combined process for preparation of an olefin oxide according to the second aspect, the epoxidation reaction conditions according to (ii) comprise fixed bed conditions.

In some preferred embodiments of the process for the regeneration of an epoxidation reactor of the first aspect or the combined process for preparation of an olefin oxide according to the second aspect, the epoxidation reaction conditions according to (ii) comprise trickle bed conditions. According to this embodiment, organic solvent, water and hydrogen peroxide and optionally additive, are mixed before entering the reactor. The olefin is introduced into the reactor as separate stream. The reaction mixture is then formed in the reactor.

In some preferred embodiments of the process for the regeneration of an epoxidation reactor of the first aspect or the combined process for preparation of an olefin oxide according to the second aspect, steps (i), (ii) and (iii) are carried out in continuous mode or in batch mode, preferably in continuous mode.

The present invention is further illustrated by the following set of embodiments and combinations of embodiments resulting from the dependencies and back-references as indicated. In particular, it is noted that in each instance where a range of embodiments is mentioned, for Example in the context of a term such as “any one of embodiments (1) to (4)”, every embodiment in this range is meant to be explicitly disclosed for the skilled person, i.e. the wording of this term is to be understood by the skilled person as being synonymous to “any one of embodiments (1), (2), (3), and (4)”. Further, it is explicitly noted that the following set of embodiments is not the set of claims determining the extent of protection, but represents a suitably structured part of the description directed to general and preferred aspects of the present invention.
1. A process for the regeneration of an epoxidation reactor, the epoxidation reactor having been used in a method for the preparation of an olefin oxide comprising:
   (i) providing an organic solvent, an olefin, an epoxidation agent and water to the reactor, which comprises a heterogeneous epoxidation catalyst in an epoxidation zone, so that a reaction mixture comprising olefin, hydrogen peroxide, water and organic solvent is formed;
   (ii) subjecting the mixture of (i) in the reactor's epoxidation zone to epoxidation conditions in the presence of the catalyst, thereby obtaining a mixture comprising water, the organic solvent and olefin oxide;
   (iii) removing the mixture comprising water, the organic solvent and olefin oxide obtained in (ii) from the reactor;
   whereby a precipitate is deposited in the reactor;
   said process for the regeneration comprising:
   (a) stopping providing organic solvent, olefin, epoxidation agent and water to the reactor;
   (b) introducing a liquid aqueous system into the reactor, wherein the liquid aqueous system comprises a chelating agent, which comprises at least three oxygen atom(s) and optionally one or more nitrogen atom(s) in its structure and which is capable of forming or forms at least three coordinative bonds via said at least three oxygen atom(s) and optionally via its one or more nitrogen atom(s) to a metal cation when forming a chelate complex with said metal cation.
2. The process for the regeneration of an epoxidation system of embodiment 1, wherein the chelating agent is selected from the group consisting of ethylene diamine tetraacetic acid (EDTA); diethylene triamine pentaacetic acid (DTPA); N-(hydroxyethyl) ethylene diamine triacetic acid (HEDTA); N-(1-carboxyethyl)-imino diacetic acid (MGDA); nitrilotriacetic acid (NTA); L-glutamic acid-N,N-diacetic acid (GLDA); aminotris(methylenephosphonic acid) (AMTP); ethanol diglycinic acid (EDG); diphosphonic acid of formula (I)

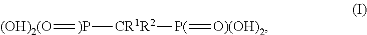

wherein R¹ and R² are independently selected from the group consisting of hydrogen atom, hydroxyl group and C1 to C5 alkyl group; and mixtures of two or more of these chelating agents.
3. The process for the regeneration of an epoxidation system of embodiment 1 or 2, wherein the chelating agent comprises, preferably is, a diphosphonic acid of formula (I)

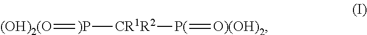

wherein R¹ and R² are independently selected from the group consisting of hydrogen atom, hydroxyl group and C1 to C5 alkyl group, wherein R¹ is preferably a hydroxyl group and R² is preferably a methyl group (1-hydroxy ethylidene-1,1-diphosphonic acid, etidronic acid, HEDP).
4. The process for the regeneration of an epoxidation reactor of any one of embodiments 1 to 3, wherein the liquid aqueous system used in (b) comprises in the range of from 0.01 to 50 weight-%, preferably in the range of from 0.02 to 25 weight-%, more preferably in the range of from 0.05 to 10 weight-% of chelating agent, preferably of diphosphonic acid of formula (I), more preferably HEDP, more preferably in the range of from 0.1 to 5 weight-% of chelating agent, preferably diphosphonic acid of formula (I), more preferably HEDP, each based on the total weight of the liquid aqueous system being 100 weight-%.
5. The process for the regeneration of an epoxidation reactor of any one of embodiments 1 to 4, wherein step (b) comprises:
   (b.1) introducing a first portion of liquid aqueous system, which comprises a chelating agent, into the reactor;
   (b.2) removing the first portion of the liquid aqueous system at least partially from the reactor.
6. The process for the regeneration of an epoxidation reactor of any one of embodiments 1 to 5, wherein step (b) comprises:
   (b.1) introducing a first portion of liquid aqueous system, which comprises a chelating agent, into the reactor;
   (b.2) removing the first portion of liquid aqueous system at least partially from the reactor;
   (b.3) optionally introducing a further portion of liquid aqueous system, which comprises chelating agent, into the reactor;
   (b.4) optionally removing the liquid aqueous system at least partially from the reactor;
   wherein steps (b.3) and (b.4) are optionally repeated for 1-10 times.
7. The process for the regeneration of an epoxidation reactor of any one of embodiments 1 to 6, wherein introducing the liquid aqueous system in step(s) (b) and/or (b.1) and/or (b.3) is carried out with a gaseous stream passing through the liquid aqueous system, wherein the gaseous stream preferably comprises at least 90 volume-%, more preferably at least 95 volume-%, more preferably at least 98 volume-% of an inert gas, based on the total volume of the gaseous stream.
8. The process for the regeneration of an epoxidation reactor of any one of embodiments 1 to 7, wherein introducing the liquid aqueous system in step(s) (b) and/or (b.1) and/or (b.3) is carried out at a temperature in the reactor in the range of from 0.1° C. to the boiling temperature of the liquid aqueous system, preferably in the range of from 0.5 to 90° C., more preferably in the range of from 5 to 50° C., more preferably in the range of from 10 to 40° C.
9. The process for the regeneration of an epoxidation reactor of any one of embodiments 1 to 8, wherein introducing the liquid aqueous system in step(s) (b) and/or (b.1) and/or (b.3) is carried out at a pressure in the reactor in the range of from 0.05 to 5.0 MPa, preferably in the range of from 0.08 to 1.0 MPa, more preferably in the range of from 0.1 to 0.15 MPa.
10. The process for the regeneration of an epoxidation reactor of any one of embodiments 1 to 9, wherein the reactor comprises one or more vertically arranged tube(s) each of a specific length (height) having a bottom end and a top end, wherein the catalyst is present in the tube(s) between a position H1 and a position H2, wherein H2 is higher up in the tube(s) than H1, and introducing the liquid aqueous system in step (b) and/or (b.1) and/or (b.3) is carried out so that the liquid level within the tube(s) is at a height of at least H2, preferably at a height in the range between H1 and H2, more preferably at a height in the range of from 80 to 5% below H2, more preferably at a height in the range of from 70 to 10% below H2, more preferably at a height in the range of from 60 to 20% below H2, more preferably at a height in the range of from 50 to 25% below H2, more preferably at a height in the range of from 40 to 30% below H2, each in relation to the distance between H1 and H2 being 100%.
11. A combined process for preparation of an olefin oxide comprising a preparation stage and a stage of regeneration of the epoxidation reactor,
   the preparation stage comprising:
   (i) providing an organic solvent, an olefin, an epoxidation agent and water to the reactor comprising an heterogeneous epoxidation catalyst in an epoxidation zone, so that a reaction mixture comprising olefin, hydrogen peroxide, water and organic solvent is formed;
   (ii) subjecting the mixture formed in (i) in the epoxidation reactor to epoxidation conditions in the presence of the catalyst, thereby obtaining a mixture comprising water, the organic solvent and olefin oxide;
   (iii) removing the mixture comprising water, the organic solvent and olefin oxide obtained in (ii) from the reactor;
   whereby a precipitate is deposited in the reactor;
   the regeneration stage comprising:
   (a) stopping introducing the mixture of (i) into the reactor;
   (b) introducing a liquid aqueous system into the reactor, wherein the liquid aqueous system comprises a chelating agent, which comprises at least three oxygen atom(s) and optionally one or more nitrogen atom(s) in its structure and which is capable of forming or forms at least three coordinative bonds via said at least three oxygen atom(s) and optionally via its one or more nitrogen atom(s) to a metal cation when forming a chelate complex with said metal cation.
12. The combined process for preparation of an olefin oxide comprising a preparation stage and a stage of regeneration of the epoxidation reactor of embodiment 11, wherein the chelating agent is selected from the group consisting of ethylene diamine tetraacetic acid (EDTA); diethylene triamine pentaacetic acid (DTPA); N-(hydroxyethyl) ethylene diamine triacetic acid (HEDTA); N-(1-carboxyethyl)-imino diacetic acid (MGDA); nitrilotriacetic acid (NTA); L-glutamic acid-N,N-diacetic acid (GLDA); aminotris(methylenephosphonic acid) (AMTP); ethanol diglycinic acid (EDG); diphosphonic acid of formula (I)

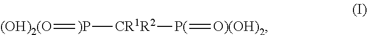

wherein R¹ and R² are independently selected from the group consisting of hydrogen atom, hydroxyl group and C1 to C5 alkyl group;
and mixtures of two or more of these chelating agents.
13. The combined process for preparation of an olefin oxide comprising a preparation stage and a stage of regeneration of the epoxidation reactor of embodiment 11 or 12, wherein the chelating agent comprises, preferably is, a diphosphonic acid of formula (I)

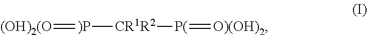

wherein R¹ and R² are independently selected from the group consisting of hydrogen atom, hydroxyl group and C1 to C5 alkyl group, wherein R¹ is preferably a hydroxyl group and R² is preferably a methyl group (1-hydroxy ethylidene-1,1-diphosphonic acid, HEDP).
14. The combined process for preparation of an olefin oxide comprising a preparation stage and a stage of regeneration of the epoxidation reactor of any one of embodiments 11 to 13, wherein the liquid aqueous system used in (b) comprises in the range of from 0.01 to 50 weight-%, preferably in the range of from 0.02 to 25 weight-%, more preferably in the range of from 0.05 to 10 weight-% of chelating agent, preferably diphosphonic acid, more preferably HEDP, more preferably in the range of from 0.1 to 5 weight-% of chelating agent, preferably diphosphonic acid, more preferably HEDP, each based on the total weight of the liquid aqueous system being 100 weight-%.
15. The combined process for preparation of an olefin oxide comprising a preparation stage and a stage of regeneration of the epoxidation reactor of any one of embodiments 10 to 12, wherein step (b) comprises:
   (b.1) introducing a first portion of liquid aqueous system, which comprises a chelating agent, into the reactor;
   (b.2) removing the first portion of the liquid aqueous system at least partially from the reactor.
16. The combined process for preparation of an olefin oxide comprising a preparation stage and a stage of regeneration of the epoxidation reactor of any one of embodiments 11 to 15, wherein step (b) comprises:
   (b.1) introducing a first portion of liquid aqueous system, which comprises a chelating agent, into the reactor;
   (b.2) removing the first portion of liquid aqueous system at least partially from the reactor
   (b.3) optionally introducing a further portion of liquid aqueous system, which comprises chelating agent, into the reactor;
   (b.4) optionally removing the further portion of liquid aqueous system at least partially from the reactor;
   wherein steps (b.3) and (b.4) are optionally repeated for 1-10 times.
17. The combined process for preparation of an olefin oxide comprising a preparation stage and a stage of regeneration of the epoxidation reactor of any one of embodiments 11 to 16, wherein introducing the liquid aqueous system in step(s) (b) and/or (b.1) and/or (b.3) is carried out with a gaseous stream passing through the liquid aqueous system, wherein the gaseous stream preferably comprises at least 90 volume-%, more preferably at least 95 volume-%, more preferably at least 98 volume-% of an inert gas, based on the total volume of the gaseous stream.
18. The combined process for preparation of an olefin oxide comprising a preparation stage and a stage of regeneration of the epoxidation reactor of any one of embodiments 11 to 17, wherein introducing the liquid aqueous system in step(s) (b) and/or (b.1) and/or (b.3) is carried out at a temperature in the reactor in the range of from 0.1° C. to the boiling temperature of the liquid aqueous system, preferably in the range of from 0.5 to 90° C., more preferably in the range of from 5 to 50° C., more preferably in the range of from 10 to 40° C.
19. The combined process for preparation of an olefin oxide comprising a preparation stage and a stage of regeneration of the epoxidation reactor of any one of embodiments 11 to 18, wherein introducing the liquid aqueous system in step(s) (b) and/or (b.1) and/or (b.3) is carried out at a pressure in the reactor in the range of from 0.05 to 5.0 MPa, preferably in the range of from 0.08 to 1.0 MPa, more preferably in the range of from 0.1 to 0.15 MPa.
20. The combined process for preparation of an olefin oxide comprising a preparation stage and a stage of regeneration of the epoxidation reactor of any one of embodiments 11 to 19, wherein the reactor comprises one or more vertically arranged tube(s) each of a specific length (height) having a bottom end and a top end, wherein the catalyst is present in the tube(s) between a position H1 and a position H2, wherein H2 is higher up in the tube(s) than H1, and introducing the liquid aqueous system in step (b) and/or (b.1) and/or (b.3) is carried out so that the liquid level within the tube(s) is at a height of at least H2, preferably at a height in the range between H1 and H2, more preferably at a height in the range of from 80 to 5% below H2, more preferably at a height in the range of from 70 to 10% below H2, more preferably at a height in the range of from 60 to 20% below H2, more preferably at a height in the range of from 50 to 25% below H2, more preferably at a height in the range of from 40 to 30% below H2, each in relation to the distance between H1 and H2 being 100%.
21. The process for the regeneration of an epoxidation reactor of any one of embodiments 1 to 10 or the combined process for preparation of an olefin oxide according to any one of embodiments 11 to 20, wherein the epoxidation agent is hydrogen peroxide, wherein the hydrogen peroxide is preferably provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484;
   and/or
   wherein the hydrogen peroxide has a pH in the range of from 0 to 3.0, preferably in the range of from 0.1 to 2.5, more preferably in the range of from 0.5 to 2.3, determined with a pH sensitive glass electrode according to AM7160;
   and/or
   wherein the hydrogen peroxide comprises from 20 to 85 weight-%, preferably from 30 to 75 weight-%, more preferably from 40 to 70 weight-% of hydrogen peroxide relative to the total weight of the aqueous hydrogen peroxide solution;
   and/or
   wherein the hydrogen peroxide is obtained or obtainable from an anthraquinone process.
22. The process for the regeneration of an epoxidation reactor of any one of embodiments 1 to 10 or the combined process for preparation of an olefin oxide according to any one of embodiments 11 to 20, wherein the organic solvent is an organic epoxidation solvent, preferably the organic solvent is selected from the group consisting of alcohol, acetonitrile, propionitrile and mixtures of two or more thereof; more preferred selected from the group consisting of alcohol, acetonitrile and mixtures of alcohol and acetonitrile; more preferred the organic solvent comprises at least an alcohol, wherein the alcohol is preferably a C1 to C5 mono alcohol or a mixture of two or more C1 to C5 alcohols, more preferred the alcohol comprises at least methanol.
23. The process for the regeneration of an epoxidation reactor of any one of embodiments 1 to 10 or the combined process for preparation of an olefin oxide according to any one of embodiments 11 to 20, wherein the olefin is a C2-C10 alkene, preferably a C2-C5 alkene, more preferably a C2-C4 alkene, more preferably ethylene (C2 alkene) or propylene (C3 alkene), more preferably propylene (C3 alkene).
24. The process for the regeneration of an epoxidation reactor of any one of embodiments 1 to 10 or the combined process for preparation of an olefin oxide according to any one of embodiments 11 to 20, wherein the mixture formed in (i) further comprise an additive, preferably selected from the group consisting of potassium salt, ammonia, ammonium salt, etidronic acid, salt of etidronic acid, and mixtures of two or more thereof; wherein the additive is selected from the group consisting of potassium dihydrogen phosphate, dipotassium hydrogen phosphate, potassium formate, potassium acetate, potassium hydrogen carbonate, etidronic acid, ammonium dihydrogen phosphate, diammonium hydrogen phosphate, ammonia and mixtures of two or more thereof, preferably form the group consisting pf potassium dihydrogen phosphate, dipotassium hydrogen phosphate, etidronic acid, ammonia and mixtures of two or more thereof.
25. The process for the regeneration of an epoxidation reactor of any one of embodiments 1 to 10 or the combined process for preparation of an olefin oxide according to any one of embodiments 11 to 20, wherein the catalyst comprising a titanium containing zeolite has a framework structure comprising Si, O, and Ti.
26. The process for the regeneration of an epoxidation reactor of any one of embodiments 1 to 10 or the combined process for preparation of an olefin oxide according to any one of embodiments 11 to 20, wherein the catalyst comprising a titanium containing zeolite comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the titanium containing zeolite.
27. The process for the regeneration of an epoxidation reactor of any one of embodiments 1 to 10 or the combined process for preparation of an olefin oxide according to any one of embodiments 11 to 20, wherein the titanium containing zeolite having a framework structure comprising Si, O, and Ti comprised in the titanium containing zeolite has ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the titanium containing zeolite having a framework structure comprising Si, O, and Ti is a titanium containing zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably an MFI framework type, or an MWW framework type; more preferred the titanium containing zeolite having a framework structure comprising Si, O, and Ti has framework type MFI; more preferably the zeolitic material having a framework structure comprising Si, O, and Ti is a titanium silicalite-1 (TS-1).
28. The process for the regeneration of an epoxidation reactor of any one of embodiments 1 to 10 or the combined process for preparation of an olefin oxide according to any one of embodiments 11 to 20, wherein the catalyst comprising a titanium containing zeolite further comprises a binder.
29. The process for the regeneration of an epoxidation reactor of any one of embodiments 1 to 10 or the combined process for preparation of an olefin oxide according to any one of embodiments 11 to 20, wherein the catalyst comprising a titanium containing zeolite is in the form of a molding, preferably in the form of an extrudate or a granule.
30. The process for the regeneration of an epoxidation reactor of any one of embodiments 1 to 10 or the combined process for preparation of an olefin oxide according to any one of embodiments 11 to 20, wherein from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the titanium containing zeolite and the binder.
31. The process for the regeneration of an epoxidation reactor of any one of embodiments 1 to 10 or the combined process for preparation of an olefin oxide according to any one of embodiments 11 to 20, wherein from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.
32. The process for the regeneration of an epoxidation reactor of any one of embodiments 1 to 10 or the combined process for preparation of an olefin oxide according to any one of embodiments 11 to 20, wherein the catalyst comprising a titanium containing zeolite, preferably the molding, comprises the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the catalyst comprising a titanium containing zeolite preferably based on the total weight of the molding and/or wherein the catalyst comprising a titanium containing zeolite, preferably the molding, comprises the titanium containing zeolite in an amount in the range of from 10 to 98 weight-%, preferably in the range of from 30 to 95 weight-%, more preferred in the in the range of from 50 to 90 weight-%, more preferred in the range of from 70 to 85 weight-%, more preferred in the range of from 75 to 80 weight-%, based on the total weight of the catalyst comprising a titanium containing zeolite, preferably based on the total weight of the molding.
33. The process for the regeneration of an epoxidation reactor of any one of embodiments 1 to 10 or the combined process for preparation of an olefin oxide according to any one of embodiments 11 to 20, wherein at least three individual feed streams are provided in (i), at least one of which is the feed with which the organic solvent is provided to the reactor, at least one of which is the feed with which the olefin is provided to the reactor and at least one of which is the feed with which the hydrogen peroxide and the water are provided to the reactor, wherein optionally, at least one further individual stream is provided to the reactor, which is the feed with which an additive is provided to the reactor.
34. The process for the regeneration of an epoxidation reactor of any one of embodiments 1 to 10 or the combined process for preparation of an olefin oxide according to any one of embodiments 11 to 20, wherein organic solvent, hydrogen peroxide, water, olefin and optional additive are mixed before entering the reactor, so that a single feed stream comprising organic solvent, hydrogen peroxide, water olefin and optional additive is provided to the reactor.
35. The process for the regeneration of an epoxidation reactor of any one of embodiments 1 to 10 or the combined process for preparation of an olefin oxide according to any one of embodiments 11 to 20, wherein the epoxidation reaction conditions according to (ii) comprise fixed bed conditions, wherein preferably organic solvent, hydrogen peroxide, water, olefin and optional additive are mixed before entering the reactor, so that a single feed stream comprising organic solvent, hydrogen peroxide, water olefin and optional additive is provided to the reactor.
36. The process for the regeneration of an epoxidation reactor of any one of embodiments 1 to 10 or the combined process for preparation of an olefin oxide according to any one of embodiments 11 to 20, wherein the epoxidation reaction conditions according to (ii) comprise trickle bed conditions, wherein preferably organic solvent, water and hydrogen peroxide and optionally additive, are mixed before entering the reactor and the olefin is introduced into the reactor as separate stream.
37. The process for the regeneration of an epoxidation reactor of any one of embodiments 1 to 10 or the combined process for preparation of an olefin oxide according to any one of embodiments 11 to 20, wherein steps (i), (ii) and (iii) are carried out in continuous mode or in batch mode, preferably in continuous mode.

The present invention is further illustrated by the following reference examples, comparative examples, and examples.

### EXAMPLES

### Reference Example 1: Preparation of a Titanium Containing Zeolite (TS-1)

In a reaction vessel, 550 kg deionised water were provided and stirred. 400 kg TPAOH (tetra-n-propylammonium hydroxide) were added under stirring. Stirring was continued for 1 h. The resulting mixture was transferred in a suitable vessel. The reaction vessel was washed twice with 2000 l deionised water in total. In the washed reaction vessel, 300 kg TEOS (tetraethoxysilane) were provided and stirred. A mixture of 80 kg TEOS and 16 kg TEOT (tetraethyl orthotitanate) was added to the 300 kg TEOS. The remaining 340 kg TEOS were added.

Subsequently, the TPAOH solution was added and the resulting mixture was stirred for another hour. Then, the reaction vessel was heated and the ethanol obtained was separated by distillation. When the internal temperature of the vessel had reached 95° C., the reaction vessel was cooled. 1143 kg water were added to the resulting suspension in the vessel and the mixture was stirred for another hour. Crystallization was performed at 175° C. within 24 h at autogenous pressure. The obtained titanium silicalite-1 crystals were separated, dried and calcined at a temperature of 500° C. in air.

The obtained powder and Walocel® were mixed in a muller and mixed for 5 min. Within 10 min, the polystyrene dispersion was continuously added. Subsequently, 15 l Ludox® AS-40 were continuously added. The resulting mixture was mixed for 5 min and polyethylene oxide was continuously added within 15 min, followed by mixing for 10 min. Then, water was added. The formable mass was extruded through a matrix having circular holes with a diameter of 1.5 mm. The obtained strands were dried in a band drier at a temperature of 120° C. for 2 h and calcined at a temperature of 550° C. in lean air (100 m³/h air/100 m³/h nitrogen). The yield was 89 kg extrudates.

For the subsequent water treatment of the extrudates, 880 kg deionised water were filled in a respective stirred vessel and the extrudates were added. At a pressure of 84 mbar, the vessel was heated to an internal temperature of from 139 to 143° C. The resulting pressure was in the range of from 2.1 to 2.5 bar. Water treatment was carried out for 36 h. The extrudates were separated by filtration, dried for 16 h at 123° C. in air, heated to a temperature of 470° C. with 2° C./min and kept at a temperature of 490° C. in air for 5 h. The yield was 81.2 kg.

### Reference Example 2: Experimental Setup for Epoxidation in a Mini-Plant

A TS-1 catalyst as obtained according to Reference Example 1 above was loaded into a reaction tube of a mini-plant with a length of 180 cm and a volume of 300 ml. The tube diameter was 0.75 inch (1.905 cm), with a wall thickness of 0.07 inch (0.19 cm). In the center of the reaction tube a smaller (0.125 inch (0.3175 cm)) tube was installed, containing thermoelements for measuring the temperature over the catalyst bed.
Feed-materials: 54 g/h Propylene (liquid)
   94 g/h aqueous H₂O₂ solution (40 weight-% H₂O₂)
Solvent: 370 g/h Methanol
Additive-solution: 4 g/h aqueous K₂HPO₄ solution (0.3 weight-% K₂HPO₄) (flow adjusted to maintain 130 micromole K⁺/(mole H₂O₂))

Propylene was stored in 50 l gas bottles, containing dip tubes, facilitating the transfer to the mini-plant by means of 25 bar nitrogen pressure. The precise amount was measured using a Bronkhorst flow meter with a 0-500 g/h range and the flow is controlled by means of a Flowserve control-valve. Hydrogen peroxide was transferred into the reactor using a Grundfos pump DME2. The amount was determined using a balance. The measurement showed liters/minute. The respective additive solution was fed to the reactor, using an hydrogen peroxide LC pump. The precise amount was determined using a balance. For feeding the methanol a Lewa pump with a range of 0-1500 ml/h was used. Feed control was accomplished using a Lewa KMM. Nitrogen was fed using a Flowserve control-valve. The amount was measured using a Bronkhorst flow meter with a range of 0-200 NI/h. “NI/h” means norm liter per hour, wherein 1 norm liter is the amount of gas, which fills 1 liter at 0° C. and 1013 mbar (see DIN 1343 from January 1990).

Methanol, propylene, aqueous hydrogen peroxide solution and aqueous K₂HPO₄ additive solution entered the reactor tube via a static mixer [0.25 inch (0.635 cm)-mixer], so that a combined feed stream was formed, wherein the feed direction was from the bottom to the top direction of the reaction tube.

The experiments were carried out at an absolute pressure of 20 bar. The temperature in the reactor was controlled to ensure a H₂O₂ conversion of approximately 90% by using a cooling jacket circuit with oil. Typical start temperature was approximately 40-45° C. Then the temperature was slowly ramped up to approximately 60-65° C. over a run-time of 600 to 700 hours. At the beginning of the run the reactor was cooled as the exothermic heat would overheat the reactor otherwise. Towards the end of the run the reactor was heated to reach a temperature of 60-65° C.

The reactor effluent was passed through a 2 micrometer filter to remove fine (catalyst) particles before it was passed into the first separator. The bottom level valve controlled a level of 25% in the first separator, while the upper pressure valve set a pressure of 20 bars over the entire upstream reaction system. The second separator was also operated at a liquid level of 25%, while the upper pressure valve reduced the pressure to 2 bars. This lower pressure served for allowing the flashing of unconverted propylene, allowing a safe sample taking, and having an additional safety buffer. The two separators had a volume of 2 liters each and were kept at a temperature of 5° C., using cooling water. A nitrogen stream of 5 NI/h was fed through the entire system (reactor→1^{st} separator→2^{nd} separator→vent-system) to maintain a sufficient gas flow in the direction of the vent to ascertain that traces of oxygen, formed by partial decomposition of H₂O₂, were flashed out and could be analyzed at the end of the vent pipe.

### Reference Example 3: Samples of Feed Precipitate

Samples of feed precipitate were obtained from a (large scale) HPPO plant, where propylene was epoxidized using hydrogen peroxide in a solvent mixture comprising water and methanol with a titanium containing zeolitic catalyst of framework type MFI, i.e. a TS-1 catalyst.

The plant comprised several reactors operated in parallel, wherein each reactor was a multitubular reactor with a bundle of vertically arranged tubes made of stainless steel, wherein selected tubes were equipped with an axially placed multi-point thermocouple with a number of equally spaced measuring points encased in a suitable thermowell. All tubes of the reactor contained strands of a heterogeneous titanium silicalite-1 (TS-1) catalyst. Through the tubes, the feed mixture was passed from the bottom to the top, i.e. in upstream mode.

The feed mixture, consisting of methanol (69.0 wt.-%), propene (11.9 wt.-%), water (11.7 wt.-%) and hydrogen peroxide (7.4 wt.-%) consisted of one single liquid phase and was fed to the multitubular reactor at room temperature (25° C.) via a feed line, divided into substreams, wherein one substream S(i) was fed via a feed line to each of the tubes. Also, the liquid reaction mixture in the reactor consisted of one single phase. The feed mixture had been made from a methanol stream, a propene stream and a stream of aqueous hydrogen peroxide solution having a hydrogen peroxide concentration of about 40 weight-%.

The heat of reaction was removed by circulating a thermostatized heat transfer medium (water/glycol mixture) on the shell side in co-current to the feed mixture. The flow rate of the heat transfer medium was adjusted so that the temperature difference between entrance and exit did not exceed 1° C. The reaction temperature referred to hereinbelow was defined as the temperature of the heat transfer medium entering the reactor shell. At the reactor exit, the pressure was controlled by a pressure regulator and kept constant at 2 MPa. The temperature of the cooling medium was chosen in such a way that the overall conversion of hydrogen peroxide at the exit of the multitubular reactor was exactly 90%.

Precipitate formed over operation of the plant and deposited in the feed lines, on filters within the feed lines if used, and in the reactor, especially at entrance into the tubes on the catalyst support, and also partially on the catalyst, and was removed during regeneration breaks of the individual reactors.

A characterization of the removed precipitate was made based on X-ray fluorescence (XRF) analysis of the solid samples, the results are shown in Table 1. Here and below, all elements are indicated for sake of simplicity without charge, but are present in the form of cations, wherein the charge is compensated by suitable anions.

TABLE 1
Precipitate Elemental Metal Composition
in collected precipitate (solid samples)

| | Precipitate Metal | | Precipitate Metal |
|---|---|---|---|
| | Concentration | | Concentration |
| Element | (weight-%) | Element | (weight-%) |
| Na | 0.476 | Cr | 0.599 |
| Mg | 0.515 | Mn | 0.216 |
| Al | 4.51 | Fe | 12.29 |
| Si | 0.788 | Ni | 0.211 |
| P | 22.55 | Cu | <0.1 |
| Cl | <0.1 | Zn | 0.59 |
| K | 9.42 | Sr | <0.1 |
| Ca | 3.65 | W | <0.1 |
| Ti | 0.112 | S | <0.1 |

### Example 1: Dissolution Testing with Water and Different Acidic Phosphates in Aqueous Solution

Samples of feed precipitate as described in Reference Example 3 were subjected to dissolution testing using the following procedure:
1. An aqueous solution of hydroxyethylidene diphosphonic acid (HEDP) with a specific strength (in the range of from 0.050 to 0.985 weight %, details as indicated in Table 2) was prepared from a 62.6 weight-% aqueous HEDP solution (Spectrum Lab Products) and deionized water. For comparison, another acidic phosphate, i.e. sodium acid pyrophosphate (SAPP, 99.1 weight-% solution from Sigma-Aldrich) was used, wherein the concentration of the aqueous solution was also adjusted to a specific strength wight deionized water (details as indicated in Table 2).
2. About 0.1 g of feed precipitate were weighted into a 20 ml vial, 10 vials in total. The correct starting precipitate quantity is indicated in Table 2.
3. 10 g of water or aqueous HEDP solution of a specific strength or aqueous SAPP solution of a specific strength were weighted into a vial.
4. The vials were shaken for at least 2 hours at room temperature (25° C.) (Thermo Scientific multi-purpose rotator, Model 2314), but checked after 1 hour to see if precipitate was dissolving.
5. A visual check was made if precipitate was still present.
6. The solutions were vacuum filtered in that:
   a. A 5-micron Millipore Durapore SVPP 47 mm filter disk was dried in a vacuum oven at about 60 C for 1 hour. After that, the filter disk was weighted and the weights recorded.
   b. The filter disk from a. was put in place in the vacuum filter.
   c. The vial was shaken and the mixture was poured on to the filter under vacuum.
   d. The vial was risen with deionized water (3 times with 5 g) and poured through filter to get all the solids out of the vial.
7. The used filter disk was dried in a vacuum oven at about 60 C for 1 hour.
8. The dried filter disk with solid on it was weighted and the weight recorded.
9. The amount of solids that had dissolved was calculated by weight difference.

All data were collected at a room temperature of about 23° C. A baseline check was first run by passing 25 g of water through each of 3 blank filters (equivalent to the phosphate solution+rinse water). The amount of solids collected on the filters was ≤0.0004 g or ≤0.4% of the initial precipitate charges used in the experiments. The contribution of the deionized water on the final results was therefore small.

The results are shown in Table 2.

TABLE 2
Results of precipitate dissolution experiment

| | | | | | | Dissolved? (visual | | | | | | |
|---|---|---|---|---|---|---|---|---|---|---|---|---|
| | | | | | | clarity check) | | | | | | |
| | | Starting | | | | | | Overnight | | | | |
| | | Precipitate | Phosphate | Phosphate | Phosphate / | | | (after | | Filter + | | Precipitate |
| | Phosphate | Quantity | Solution | Concentration | (Al + Fe) | after 1 | after 2 | about | Filter | solids | Solids | dissolved |
| No. | Type | (g) | (g) | (weight-%) | (m/m) | hour? | hours? | 14 hours) | (g) | (g) | (g) | (weight-%) |
| 1 | none | 0.1011 | 10.018 | 0.000 | 0.000 | N | N | N | 0.1311 | 0.2142 | 0.0831 | 17.8 |
| 2 | SAPP | 0.1009 | 10.014 | 0.050 | 0.073 | N | N | N | 0.1208 | 0.203 | 0.0822 | 18.5 |
| 3 | SAPP | 0.101 | 10.0039 | 0.495 | 0.718 | N | N | N | 0.1416 | 0.214 | 0.0724 | 28.3 |
| 4 | SAPP | 0.1004 | 10.0075 | 0.990 | 1.445 | N | N | — | 0.1269 | 0.2064 | 0.0795 | 20.8 |
| 5 | HEDP | 0.101 | 10.0052 | 0.010 | 0.016 | N | N | N | 0.1383 | 0.2186 | 0.0803 | 20.5 |
| 6 | HEDP | 0.1003 | 10.0147 | 0.053 | 0.083 | N | N | N | 0.1297 | 0.1814 | 0.0517 | 48.5 |
| 7 | HEDP | 0.1022 | 10.008 | 0.522 | 0.806 | N | Y | Y | 0.1355 | 0.1392 | 0.0037 | 96.4 |
| 8 | HEDP | 0.1004 | 10.0055 | 1.043 | 1.640 | Y | Y | — | 0.1288 | 0.1316 | 0.0028 | 97.2 |
| 9 | none | 0.1004 | 10.0122 | 0.000 | 0.000 | N | N | — | 0.1284 | 0.2242 | 0.0958 | 4.6 |
| 10 | HEDP | 0.1028 | 10.0141 | 1.043 | 1.269 | Y | Y | — | 0.1282 | 0.1351 | 0.0069 | 93.3 |

“N” no
“Y” yes
“—“ not determined

In the cases where neat water was the solvent, only 4.6 and 17.8 weight-% of the solids were dissolved. When SAPP was added to the water at up to 1 weight-%, the result was not greatly improved, with dissolution in the 20 weight-% range. Incremental addition of HEDP to the water on the other hand significantly improved dissolution. Already with 0.05 weight-% HEDP, the solids dissolution was more than twice the amount of what was dissolved with 0.05 weight-% SAPP (see Table 2, no. 2 versus no. 6). With both 0.5 and 1.0 weight-% HEDP solutions, solids dissolution was in the mid-90 weight-% range (see Table 2, no. 7, 8 and 10).

Increasing the concentration of HEDP in the aqueous solution hastened dissolution by visual observation. The mixture made with aqueous solution having 0.5 weight-% HEDP was still quite cloudy with solids after 1 hour of shaking, while the solids appeared to be near fully dissolved when an aqueous solution with 1 weight-% HEDP was used. The mixture made with aqueous solution having 0.5 weight-% HEDP solution cleared up after 2 hours of shaking. It was later determined that precipitate (0.1 g) mixed with aqueous solution having 5 weight-% HEDP (10 g) dissolved in about 2 minutes when shaking gently by hand and even sooner when the HEDP concentration in the aqueous solution was increased to 10 weight-%.

The HEDP or SAPP to metal in the precipitate ratio was calculated to see if there was a relationship to dissolution. Based on the concentrations indicated in Table 1 in Reference Example 3, the 2 primary metals in precipitate, Al and Fe, were used as basis. There was more than 1 mole of phosphate/mole of precipitate metal in the 1 weight-% HEDP cases that easily dissolved the precipitate. The ratio was 0.8 for the 0.5 weight-% HEDP case that was capable of dissolving the precipitate albeit more slowly. Targeting at least a 1:1 molar HEDP to precipitate metals should be a reasonably good guideline for determining the minimum quantity of HEDP required for cleaning.

A characterization of the original precipitates and the residue remaining after dissolving with the 1% HEDP is shown in Table 3, wherein the data on the precipitate metal concentration before treatment are the same as indicated in Table 1, the residue analyzed was from no. 10 as listed in Table 2. The data were based on X-ray fluorescence (XRF) analysis of the solid samples.

TABLE 3
Precipitate Elemental Metal Composition Before and After HEDP Dissolution

| | Precipitate Metal | Precipitate Metal | |
|---|---|---|---|
| | Concentration* before | Concentration after | |
| | treatment with 1.043 | treatment with 1.043 | Amount of Element |
| | weight-% aq. HEDP | weight-% aq. HEDP** | Removed from |
| Element | (weight-%) | (weight-%) | Precipitate (%) |
| Na | 0.476 | 1.65 | 77 |
| Mg | 0.515 | 1.09 | 86 |
| Al | 4.51 | 5.49 | 92 |
| Si | 0.788 | 16.17 | −38 |
| P | 22.55 | 0.392 | 100 |
| Cl | <0.1 | 0.766 | — |
| K | 9.42 | 1.2 | 99 |
| Ca | 3.65 | 1.57 | 97 |
| Ti | 0.112 | 2.89 | −73 |
| Cr | 0.599 | 2.17 | 76 |
| Mn | 0.216 | 0.332 | 90 |
| Fe | 12.29 | 22.43 | 88 |
| Ni | 0.211 | 0.365 | 88 |
| Cu | <0.1 | 0.222 | — |
| Zn | 0.59 | 1.11 | 87 |
| Sr | <0.1 | 0.93 | — |
| W | <0.1 | 0.48 | — |
| S | <0.1 | 0.283 | — |

*data are the same as indicated in Table 1
**no. 10 from Table 2
“—” not determined

The leftover insolubles after treatment with 1.043 weight-% aqueous HEDP solution were mainly composed of Fe, Si and Al.

Of the most concentrated elements in the starting material, the aqueous HEDP solution dissolved about 90% of the Al, Fe, and Ca according to the mass balance in the far right column in Table 3. The mass balance is based on the starting and residual solids weights and concentrations. The HEDP removed virtually all of the P and K.

The Si and Ti concentrations increased prominently. The mass balance showed these components were very insoluble. The form of this Si and Ti was unknown but the observation was encouraging since TS-1 epoxidation catalyst is composed of Si and Ti. A wash medium must not damage the catalyst or the catalyst's performance for it to be a viable candidate.

### Example 2: Determination of Effect of Washing with HEDP on Process Vessels

In order to determine if metals might leach off of the process walls to a significant level during a reactor wash, a compatibility check was run. There was a concern that metals could be deposited on the catalyst rendering a decrease in its performance as well as a very minor concern of equipment corrosion. A 1 weight-% aqueous solution of HEDP was contacted with passivated stainless steel (316SS) for a week after which metal concentrations were measured in the solution. This following procedure that was followed:
1. An 8 oz. (227 g) jar and seven 20 ml jars were cleaned:
   a. Each jar was filled with about 5% HNO₃ and let sit overnight then
   b. The acid was discarded and the jars were washed with deionized water 3 times.
2. An aqueous 1 weight-% solution of HEDP was made in the 8 oz. (227 g) jar:
   a. 3.7 g of nominal 60 weight-% aqueous solution of HEDP were added to the jar,
   b. Deionized water was added to make up to a total weight of 220 g, then
   c. Shaking was done for mixing.
3. Six of the 20 ml jars were filled about 50% full with the aqueous 1 weight-% solution of HEDP, swirl, then the contents were discarded. The jars were left drip dry.
4. Each of the six rinsed 20 ml jars was filled with about 17 g of aqueous 1 weight-% solution of HEDP. Weights were recorded.
5. Four ¼″ (6.350 mm) passivated Swagelok port connector tubing fittings were put into the last empty 20 ml jar. Passivation consisted of treatments with a series of solutions: trisodium phosphate/sodium metasilicate, sodium hydroxide, then nitric acid. Rinsing and swirling was done with deionized water 3 times. Methanol was added for covering. Shaking was done gently for 15 minutes then the methanol was discarded and the residual methanol was allowed to evaporate in the hood.
6. One of the fittings was added to each of four jars containing HEDP solution. The port connector fittings each weighted about 4.3 g and had about 9.3 cm² of surface area.
7. The jars were put into an oven that was preheated to 60 C. The lids were loose so that pressure could be relieved. The time was recorded.
8. After 24 hours, two of the jars containing a fitting were removed from the oven. The fittings were carefully removed using a plastic spatula cleaned with 5 weight-% HNO₃ aqueous solution and rinsed with reverse osmosis water. The jars were labeled as “1% HEDP solution from 316SS contact, 24 hrs @60 C”
9. After a total of 1 week, the other jars were removed from the oven. The fittings were carefully removed using a plastic spatula cleaned with 5 weight-% HNO₃ aqueous solution and rinsed with Reverse osmosis water. These jars were labelled as “1% HEDP solution from 316SS contact, 168 hrs @60 C”. The other jars were labelled as “1% HEDP solution, 168 hrs @60 C”.
10. The metal concentrations were analysed in the solutions using inductively coupled plasma mass spectrometry (ICP-MS).

The results of the metals leaching test are shown in Table 4. Li, Be, V, Ga, Se, Rb, Sr, Nb, Rh, Ag, Cd, Sn, Cs, Ba, Pb, and Bi were all non-detectable (<5 ppb). A very minor increase (difference between solution in contact with metal for 168 hrs vs. 1% HEDP only) in two 316SS components was observed, <100 ppb of Ni and Cr. The increase in Fe was larger, but was still a rather small 390 ppb increase after 24 hours and 570 ppb after 168 hours. The rate of Fe increase dropped off significantly after 24 hours, perhaps indicating the HEDP was completing passivation of the surface.

The Fe removed, as calculated by the solution mass, change in solution concentration, and fitting surface area, equated to 0.01 g/m²/week of metal. This confirmed there was no corrosion concern.

TABLE 4
Average Solution Metals Concentrations
from HEDP - 316SS Compatibility Test

| | 1% HEDP | 1% HEDP w/316 | 1% HEDP w/316 |
|---|---|---|---|
| | 168 hr | ss 24 hr | ss 168 hr |
| | ng/ml | ng/ml | ng/ml |
| Mg | 48 | 60 | 48 |
| Al | 158 | 94 | 154 |
| K | 22 | 39 | 25 |
| Ti | 25 | 24 | 24 |
| Cr | <5 | 33 | 69 |
| Mn | 11 | 32 | 28 |
| Fe | 14 | 402 | 583 |
| Co | <5 | <5 | 6 |
| Ni | <5 | 32 | 48 |
| Cu | <5 | 12 | 21 |
| Zn | 6 | 39 | 35 |
| As | 18 | 18 | 17 |
| Zr | 10 | <5 | <5 |
| Mo | <5 | 8 | 11 |
| Sb | 12 | 13 | 14 |

### Example 3: TS-1 Catalyst Performance after HEDP Treatment

A test-run was carried out in a mini-plant as described in Reference Example 2 to further confirm that the proposed HEDP treatment of the TS-1 catalyst does not have an unfavorable effect on the catalyst performance at HPPO reaction conditions. For this purpose a dual reactor system was used. While one reactor was loaded with fresh untreated TS-1 catalyst, the second reactor was loaded with the same catalyst and then a 1 weight-% aqueous solution of HEDP was fed over the catalyst bed for 24 hours at a temperature of 50° C. at a feed-rate of 1 liter/hour. Following that treatment the catalyst was washed with demineralized water for 2 hours at a temperature of 50° C. at a feed-rate of 1 liter/hour.

After this pre-treatment the regular HPPO reaction was started at the standardized catalyst screening conditions as described in Reference Example 2 in parallel in both reactors. The test run was conducted for a time period of 600 hours. The result of this screening experiment is shown in FIG. 1.

No significant performance differences between the treated and untreated catalyst were observed at these conditions during the entire test-run further supporting that the HEDP-treatment does not have a negative effect on the catalyst activity.

To evaluate the effect on catalyst composition and mechanical stability samples of the treated and untreated TS-1 catalyst were analysed after the test-run. No titanium was lost from the HEDP treated catalyst in comparison to the untreated material.

### Example 4: Evaluation of Mechanical Stability

Due to the potential risk associated with softening the catalyst by treating it with HEDP, follow-up lab tests were performed and Crush strength analyses were made.

Treated catalyst samples were prepared using a TS-1 catalyst according to Reference Example 1. In a 20-ml glass vial, 1 g samples of TS-1 were combined with 10 g of water or 1 weight-% aqueous HEDP solution. Water treatment was chosen as a comparison. A set of samples was shaken for 24 hours then allowed to sit stagnant for 3 days at ambient temperature (23° C.). In another case, the mixtures were left stagnant for 3 days. A sample of fresh, untreated catalyst was used for reference as well as a sample of spent catalyst in accordance with Reference Example 3, wherein the epoxidation of Reference Example 3 had been carried out for 1044 hours. The catalyst had been used in 5 previous runs for a sum total of 3679 hours of operation, and had been calcined after each previous run to restore activity before being used in the next run.

Samples treated with HEDP solution were washed with deionized water at the end of treatment. All samples were then dried in an oven at 80° C. overnight.

The crush strength of 15 random particles for each treatment was measured with an Instron Model 5543-C7585 load frame. The travel speed of the crosshead was set at 2 mm/min. The instrument load cell was calibrated.

The ends of the catalyst particles for which treatment included shaking were noticeably more rounded and there was a small amount of catalyst dust present in the jars, but they were well intact otherwise. All the other catalyst samples visually appeared to be in good condition. There was no discernible difference in crush strength for any of the catalysts tested. The results are shown in Table 5.

TABLE 5
Follow-up study of treatment effect on catalyst crush strength

| | | Mean | Median | |
|---|---|---|---|---|
| | | Crush | Crush | Standard |
| | Catalyst &Treatment | Strength | Strength | Deviation |
| Solution | Duration | (N) | (N) | (N) |
| None | Fresh Catalyst - untreated | 8.7 | 7.3 | 4.7 |
| none | spent catalyst - untreated | 7.0 | 7.1 | 3.5 |
| Water | spent catalyst - 3 days stagnant | 9.1 | 8.3 | 4.1 |
| Water | spent catalyst - Shaken 24 hours | 8.2 | 8.0 | 3.8 |
| | then 3 days stagnant | | | |
| 1% | spent catalyst - 3 days stagnant | 6.8 | 4.7 | 4.5 |
| HEDP | | | | |
| 1% | spent catalyst - Shaken 24 hours | 8.8 | 7.7 | 3.9 |
| HEDP | then 3 days stagnant | | | |

### Example 5: Tube Cleaning Simulation

Further experiments were made in order to scale-up the lab precipitation results and to find out how much of the catalyst had to be exposed to the washing solution and whether circulation improved the removal of precipitate.

### Example 5a: Aqueous HEDP Solution Only in the Bottom Region

In a HPPO plant with a vertically arranged multitubular reactor, the precipitate was mostly concentrated in the bottom head of the reactor and did not penetrate deeply into the tubes. Therefore, an experiment with a vertically arranged tube reactor was made where the aqueous HEDP solution was introduced into the bottom head with sufficient volume to reach only the lowest 10% of the catalyst, i.e. the catalyst situated in the bottom region of the tubes. In a lab simulation, first an approach was made with an “ebb and flow” of solution. Glass tubes (½″ inner diameter (12.7 mm)) were made that had a coarse glass frit on the bottom. The tubes were loaded with TS-1 catalyst according to Reference Example 1 and a thin layer of a feed precipitate sample as described in Reference Example 3. The tubes were dried in an 80° C. oven then were weighed. The tubes were then vertically immersed in a jar containing a sufficient level of 1 weight-% HEDP aqueous solution to cover the catalyst once it filled the tube through the frit. Tubes were lifted, allowed to drain, and then re-immersed at various periods of time to simulate emptying and re-filling plant exchanger tubes. At the conclusion, the tubes were rinsed with 10 cm³ of deionized water from the top of the tubes. They were then dried in an 80° C. oven and re-weighted.

Three tubes were used for this experiment and all were immersed in the same jar of 1 weight-% HEDP aqueous solution. The total immersion period was 24 hours. One tube was left in solution the entire time, one was drained and refilled every 4 hours, and the last was drained and refilled every hour. The weights are given in Table 6. About 30% of precipitate was removed just by leaving it stagnant for 24 hours, but, refreshing the HEDP solution in the tubes was effective at increasing dissolution. For the tube that was drained every hour, the removal was 76 weight-%.

TABLE 6
Results of intermittent immersion tube simulation

| | Tube 1 | Tube 2 | Tube 3 |
|---|---|---|---|
| | Stagnant | Drained | Drained |
| Treatment | 24 hrs | every 4 hrs | every hr |
| Initial Precipitate Wt. (g) | 0.4913 | 0.4896 | 0.4926 |
| Initial Total Tube Wt. (g) | 28.812 | 28.132 | 28.378 |
| Final Total Tube Wt. (g) | 28.663 | 27.899 | 28.006 |
| Wt. Change (g) | 0.149 | 0.233 | 0.372 |
| Weight-% of Precipitate | 30 | 48 | 76 |
| Removed | | | |
| # of Refreshes | 0 | 6 | 24 |

Visual examination of the tubes showed that there was more movement downward of darker-colored, insoluble precipitate particles in the tube that was refreshed hourly. The frit was effective at containing the particles within the tubes so there was no migration of precipitate outside the tubes other than what dissolved.

### Example 5b: Induced Circulation

To further simplify tube refreshment, exploration of sparging the bottom head of the exchanger with nitrogen was explored in order to simulate an induced circulation. A 1″ (2.54 cm) inner diameter tube was fabricated that contained a flared “bell” on either end. The bottom bell was used to collect nitrogen gas supplied by tubing metered with a valved rotameter. The top bell was used to allow good disengagement of the gas upon exit. A metal screen was put at the bottom of the straight section to support the tube contents. It was found during testing with water that this screen needed fairly large holes, 3/32″ (2.38 mm) perforations, to allow gas to easily pass through it. To retain the small TS-1 catalyst particles (1.5 mm diameter extrudate), a layer of ¼″ (6.350 mm) Denstone alumina balls were laid on top of the screen. Then a layer of catalyst was added followed by a thin layer of precipitate and another layer of catalyst up to the top of the straight section. The tube was dried in an 80° C. oven then it was weighed. About 900 ml of 1 weight-% HEDP aqueous solution was added to a 1000 ml beaker. The loaded glass tube was slowly lowered down into the solution with the bell centered over the end of the nitrogen tube that was at the bottom of the beaker.

Two tests were run with this set-up. The first consisted of maintaining a constant flow of nitrogen for a given period of time. A flow of 0.5 scfh (0.24 l/min) was chosen as an appropriate flow. The reason for choosing this flow is that flows significantly greater than this during tests with water caused some upward movement of the catalyst. At sufficiently high nitrogen flow (2+ scfh range), ejection of small catalyst clumps from the top can occur. Movement was most noticeable when first initiating gas flow. This test was run for 19 hours. As in the intermittent immersion test, there was some migration of precipitate downward. Overall, it appeared to do an effective job as only a small amount of the precipitate was visible by the end of the test.

The second sparging test was done with intermittent flow of nitrogen. The flow of nitrogen was again 0.5 scfh (0.24 l/min). After every 5 minutes, the nitrogen flow was turned off for about 15 seconds then restarted. The test ran over a 2 hour period and consisted of 24 of these cycles. From visual inspection, it was apparent that there was more movement and mixing within the tube by this method. After 15 minutes, a trail of precipitate dust that migrated downward was visible in the bell on the left side. The solution was quite cloudy within about 30 minutes. This was presumably because of catalyst dust that was washed off of the catalyst (this cloudiness was also observed during water tests where there was no precipitate present in the tube) and another sign of improved turnover in the tube. A few black specks of insoluble precipitate were visible in the tube at the end of the test but the normally soluble gray precipitate could not be seen. The initial charge of precipitate to the tube was 1.02 g. The tube weight change was a loss of 0.9 g. Solids that settled out in the beaker amounted to 0.08 g and appeared to be a combination of catalyst dust and insoluble precipitate (some light and some dark particles). If this amount was added back in with the tube contents, the precipitate dissolution was 80%. Considering the test only ran for 2 hours, this was the most effective technique tried.

### SHORT DESCRIPTION OF THE FIGURES

FIG. 1 shows a performance comparison of HEDP washed TS-1 catalyst vs. untreated TS-1 catalyst.

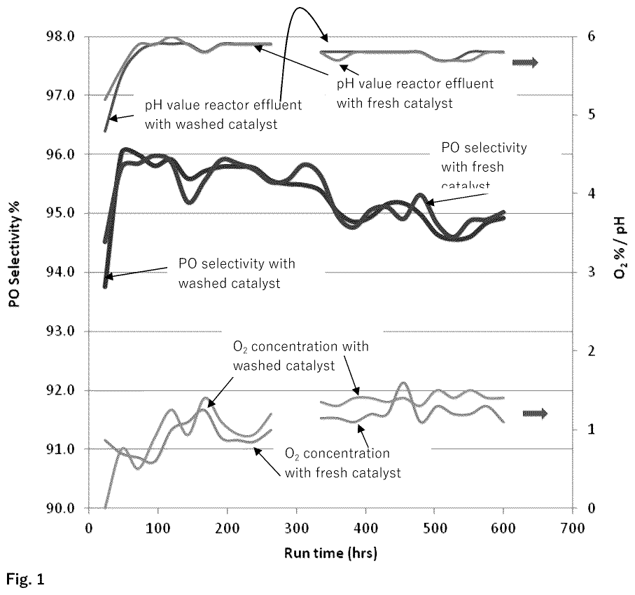

### CITED LITERATURE

WO 2016/128538 A1
WO 2015/010994 A1

1.-14. (canceled)

15. A process for the regeneration of an epoxidation reactor, the epoxidation reactor having been used in a method for the preparation of an olefin oxide comprising:
(i) providing an organic solvent, an olefin, an epoxidation agent and water to the reactor, which comprises an heterogeneous epoxidation catalyst in an epoxidation zone, so that a reaction mixture comprising olefin, hydrogen peroxide, water and organic solvent is formed;
(ii) subjecting the mixture of (i) in the reactor's epoxidation zone to epoxidation conditions in the presence of the catalyst, thereby obtaining a mixture comprising water, the organic solvent and olefin oxide; and
(iii) removing the mixture comprising water, the organic solvent and olefin oxide obtained in (ii) from the reactor;
whereby a precipitate is deposited in the reactor;
the process for the regeneration comprising:
(a) stopping providing organic solvent, olefin, epoxidation agent and water to the reactor; and
(b) introducing a liquid aqueous system into the reactor, wherein the liquid aqueous system comprises a chelating agent, which comprises a diphosphonic acid of formula (I) 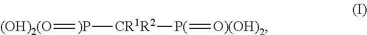
wherein R¹ and R² are independently selected from the group consisting of hydrogen atom, hydroxyl group and C1 to C5 alkyl group.

16. The process for the regeneration of an epoxidation system of claim 15, wherein the chelating agent is a diphosphonic acid of formula (I).

17. The process for the regeneration of an epoxidation system of claim 15, wherein R¹ is a hydroxyl group and R² is a methyl group (1-hydroxy ethylidene-1,1-diphosphonic acid, HEDP).

18. The process for the regeneration of an epoxidation reactor of a claim 15, wherein the liquid aqueous system used in (b) comprises in the range of from 0.01 to 50 weight-% of the chelating agent.

19. The process for the regeneration of an epoxidation reactor of claim 15, wherein step (b) comprises:
(b.1) introducing a first portion of liquid aqueous system, which comprises a chelating agent, into the reactor;
(b.2) removing the first portion of the liquid aqueous system at least partially from the reactor;
wherein step (b) comprises:
(b.3) introducing a first portion of liquid aqueous system, which comprises a chelating agent, into the reactor;
(b.4) removing the first portion of liquid aqueous system at least partially from the reactor
(b.5) optionally introducing a further portion of liquid aqueous system, which comprises chelating agent, into the reactor;
(b.6) optionally removing the further portion of liquid aqueous system at least partially from the reactor;
wherein steps (b.3) and (b.4) are optionally repeated for 1-10 times.

20. The process for the regeneration of an epoxidation reactor of claim 15, wherein introducing the liquid aqueous system in step(s) (b) and/or (b.1) and/or (b.3) is carried out with a gaseous stream passing through the liquid aqueous system, wherein the gaseous stream comprises at least 90 volume-% of an inert gas, based on the total volume of the gaseous stream.

21. The process for the regeneration of an epoxidation reactor of claim 15, wherein introducing the liquid aqueous system in step(s) (b) and/or (b.1) and/or (b.3) is carried out at a temperature in the reactor in the range of from 0.1° C. to the boiling temperature of the liquid aqueous system.

22. The process for the regeneration of an epoxidation reactor of claim 15, wherein introducing the liquid aqueous system in step(s) (b) and/or (b.1) and/or (b.3) is carried out at a pressure in the reactor in the range of from 0.05 to 5.0 MPa.

23. The process for the regeneration of an epoxidation reactor of claim 15, wherein the reactor comprises one or more vertically arranged tube(s) each of a specific length (height) having a bottom end and a top end, wherein the catalyst is present in the tube(s) between a position H1 and a position H2, wherein H2 is higher up in the tube(s) than H1, and introducing the liquid aqueous system in step (b) and/or (b.1) and/or (b.3) is carried out so that the liquid level within the tube(s) is at a height in the range between H1 and H2, each in relation to the distance between H1 and H2 being 100%.

24. A combined process for preparation of an olefin oxide comprising a preparation stage and a stage of regeneration of the epoxidation reactor,
the preparation stage comprising:
(i) providing an organic solvent, an olefin, an epoxidation agent and water to the reactor comprising an heterogeneous epoxidation catalyst in an epoxidation zone, so that a reaction mixture comprising olefin, hydrogen peroxide, water and organic solvent is formed;
(ii) subjecting the mixture formed in (i) in the epoxidation reactor to epoxidation conditions in the presence of the catalyst, thereby obtaining a mixture comprising water, the organic solvent and olefin oxide;
(iii) removing the mixture comprising water, the organic solvent and olefin oxide obtained in (ii) from the reactor;
whereby a precipitate is deposited in the reactor;
the regeneration stage comprising:
(a) stopping introducing the mixture of (i) into the reactor;
(b) introducing a liquid aqueous system into the reactor, wherein the liquid aqueous system comprises a chelating agent, which comprises a diphosphonic acid of formula (I) 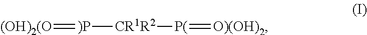
wherein R¹ and R² are independently selected from the group consisting of hydrogen atom, hydroxyl group and C1 to C5 alkyl group, wherein R¹ is a hydroxyl group and R² is a methyl group (1-hydroxy ethylidene-1,1-diphosphonic acid, HEDP).

25. The process for the regeneration of an epoxidation reactor of claim 15, wherein at least three individual feed streams are provided in (i), at least one of which is the feed with which the organic solvent is provided to the reactor, at least one of which is the feed with which the olefin is provided to the reactor and at least one of which is the feed with which the hydrogen peroxide and the water are provided to the reactor, wherein optionally, at least one further individual stream is provided to the reactor, which is the feed with which an additive is provided to the reactor.

26. The process for the regeneration of an epoxidation reactor of the combined process for preparation of an olefin oxide according to claim 24, wherein at least three individual feed streams are provided in (i), at least one of which is the feed with which the organic solvent is provided to the reactor, at least one of which is the feed with which the olefin is provided to the reactor and at least one of which is the feed with which the hydrogen peroxide and the water are provided to the reactor, wherein optionally, at least one further individual stream is provided to the reactor, which is the feed with which an additive is provided to the reactor.

27. The process for the regeneration of an epoxidation reactor of claim 15, wherein organic solvent, hydrogen peroxide, water olefin and optional additive are mixed before entering the reactor, so that a single feed stream comprising organic solvent, hydrogen peroxide, water, olefin and optional additive is provided to the reactor.

28. The process for the regeneration of an epoxidation reactor of the combined process for preparation of an olefin oxide according to claim 24, wherein organic solvent, hydrogen peroxide, water olefin and optional additive are mixed before entering the reactor, so that a single feed stream comprising organic solvent, hydrogen peroxide, water, olefin and optional additive is provided to the reactor.

29. The process for the regeneration of an epoxidation reactor of claim 15, wherein the epoxidation reaction conditions according to (ii) comprise fixed bed conditions, wherein organic solvent, hydrogen peroxide, water, olefin and optional additive are mixed before entering the reactor, so that a single feed stream comprising organic solvent, hydrogen peroxide, water olefin and optional additive is provided to the reactor or wherein the epoxidation reaction conditions according to (ii) comprise trickle bed conditions, wherein organic solvent, water and hydrogen peroxide and optionally additive, are mixed before entering the reactor and the olefin is introduced into the reactor as separate stream.

30. The process for the regeneration of an epoxidation reactor of the combined process for preparation of an olefin oxide according to claim 24, wherein the epoxidation reaction conditions according to (ii) comprise fixed bed conditions, wherein organic solvent, hydrogen peroxide, water, olefin and optional additive are mixed before entering the reactor, so that a single feed stream comprising organic solvent, hydrogen peroxide, water olefin and optional additive is provided to the reactor or wherein the epoxidation reaction conditions according to (ii) comprise trickle bed conditions, wherein organic solvent, water and hydrogen peroxide and optionally additive, are mixed before entering the reactor and the olefin is introduced into the reactor as separate stream.

31. The process for the regeneration of an epoxidation reactor of claim 15, wherein steps (i), (ii) and (iii) are carried out in continuous mode or in batch mode.

32. The process for the regeneration of an epoxidation reactor of the combined process for preparation of an olefin oxide according to claim 24, wherein steps (i), (ii) and (iii) are carried out in continuous mode or in batch mode.
